(19) United States
(12) Patent Application Publication
(10) Pub. No.: US 2023/0087240 A1
(43) Pub. Date: Mar. 23, 2023

(54) COOLING GARMENT

(71) Applicant: Delta T AZ LTD, Tel Aviv (IL)

(72) Inventors: Tzach Yitshak FENIGSHTEIN, Tel Aviv (IL); Yana Anna LEV-LUBARSKY, Tel Aviv (IL)

(73) Assignee: Delta T AZ LTD, Tel Aviv (IL)

(21) Appl. No.: 17/802,561

(22) PCT Filed: Feb. 16, 2021
(86) PCT No.: PCT/IB2021/051304

§ 371 Date: Aug. 26, 2022

(30) Foreign Application Priority Data
Feb. 26, 2020 (IL) 272925

(51) Int. Cl. A41D 13/005; A61F 7/00

(52) U.S. Cl. CPC A41D 13/0056; A61F 7/00; A61F 2007/0068

(57) ABSTRACT

A cooling garment including an article of clothing; and a cooling system associated with the article of clothing, which includes (a) a fabric portion incorporated into or integrated within the article of clothing; and (b) a fluid supply and dispensing apparatus for supplying and dispensing fluid to the fabric portion. The apparatus includes (i) at least one fluid bladder for holding fluid; (ii) a fluid pumping mechanism; and (iii) tubing having a tubing outlet for conveying fluid from the bladder and dispensing the fluid from the tubing outlet to the fabric portion. The clothing is configured to hold the fabric portion snugly to the body of the wearer and the fabric portion is disposed adjacent at least one superficial blood vessel so as to dispense the fluid specifically to the fabric portion adjacent the at least one superficial blood vessel and not to other areas of the clothing.

### FIELD OF THE INVENTION

The present invention relates to sport clothes, in particular a garment for cooling an active person.

### BACKGROUND OF THE INVENTION

Overheating during exercising is uncomfortable, can impair performance, and is potentially even life threatening. To ameliorate this issue, garments, in particular shirts, have been designed with cooling features.

It is believed that the following publications represent the relevant technology in the field: JP 2017020140 (Kunihiko, 2017-01-26); US 2010/018,682 (Goldmann et al., 2010-01-28); US 8,443,463 (Paull, 2011-11-24); US 5,715,533 (Stein, 1998-02-10); CN 107713089 (Yu Wenming, 2018-02-23); CN 105433460 (Chen Weiyu, 2016-03-30); US 5,940,880 (Phillips, 1999-08-24); KR 20040103828 (Suk, 2004-12-09); US 7,721,349 (Strauss, 2010-05-25); and US 2009/235680 (Serano, 2009-09-24). These publications are incorporated herein by reference in their entirety.

### SUMMARY OF THE INVENTION

The present invention relates to a garment designed to cool the wearer. It is a particular feature of the cooling garment that it takes advantage of blood flow in the body to help in cooling the wearer.

In accordance with one aspect of the present invention there is provided a cooling garment for cooling a wearer. The garment includes an article of clothing; and a cooling system associated with the article of clothing. The cooling system includes a fabric portion incorporated into or integrated within the article of clothing; and a fluid supply and dispensing apparatus for supplying and dispensing fluid to the fabric portion. The fluid supply and dispensing apparatus includes: (a) at least one fluid bladder for holding fluid; (b) a fluid pumping mechanism; and (c) tubing having a tubing outlet for conveying fluid from the bladder and dispensing the fluid from the tubing outlet to the fabric portion. The clothing is configured to hold the fabric portion snugly to the body of the wearer and the fabric portion is disposed adjacent at least one superficial blood vessel. As such, fluid is dispensed specifically to the fabric portion adjacent the at least one superficial blood vessel and not to other areas of the clothing.

The at least one superficial blood vessel may include any one or combination of: an axillary vein, a basilic vein; a brachial vein, a median cubital vein; a cephalic vein; and a subclavian vein. The at least one superficial blood vessel may include any one or combination of: an ulnar artery; a radial artery; and a brachial artery.

The fabric portion may be made of the same material as the clothing and completely homogenous therewith. Alternatively, the fabric portion may be made of a different material than the clothing (i.e. not the same material as the clothing). The fabric portion may be made of a different material than the remainder of clothing. The fabric portion may be made of a different thickness than the remainder of clothing. The fabric portion may be made of a material having a different porosity than the remainder of clothing. The fabric portion may be made of a material having a different tortuosity than the remainder of clothing. The fabric portion may be rectangular. The fabric portion may be 10-15 cm long and 2-4 cm wide.

The tubing may have at least one fluid outlet disposed at an upper end of the fabric portion. The clothing may be a shirt. The clothing may be constituted by an arm-sleeve or pair of arm-sleeves, and not an entire shirt.

The fluid pumping mechanism may include an electric pump. The fluid pumping mechanism may include a pressurized container. The fluid pumping mechanism may include a syringe arrangement. The fluid pumping mechanism may include a hand pump. The fluid pumping mechanism may include a wicking material.

The garment may include a sensor and a controller configured to automatically operate the fluid pumping mechanism based on an environmental parameter and/or a biological parameter. The environmental parameter may be an ambient temperature. The environmental parameter may be skin temperature.

The clothing may include a tightening mechanism to hold the fabric portion snugly to the wearer.

The fluid supply and dispensing apparatus may include cylindrically shaped tubing. The tubing may include a portion that is streamlined.

In accordance with another aspect of the present invention there is provided a method of cooling a wearer of a garment. The method includes supplying and dispensing fluid to an article of clothing of the garment while being worn by a wearer. The fluid is dispensed to a portion of the clothing that is held snuggly and adjacently to at least one superficial blood vessel. As such, fluid is dispensed specifically to the fabric portion adjacent the at least one superficial blood vessel and not to other areas of the clothing.

The method may further include automatically controlling delivery of the fluid based on an environmental parameter and/or a biological parameter. The automatic controlling of the delivery of the fluid may be based on ambient temperature. The automatic controlling of the delivery of the fluid may be based on skin temperature.

Superficial veins are commonly visible through the skin. Such veins, especially if located below the level of the heart, may bulge outward. Superficial veins typically become more prominent when exercising, in particular when lifting weights or during other strength workouts.

The terms “prominent blood vessel” and/or “superficial blood vessel” refer to blood vessels that run close to the surface of the skin and/or situated so as to be readily visible during exercise, and may be used interchangeably herein the specification and claims.

Accordingly, prominent blood vessels / superficial blood vessels include any one or a combination of: an axillary vein, a basilic vein; a median cubital vein; a cephalic vein; a brachial vein and a subclavian vein. The prominent blood vessels / superficial blood vessels further or alternatively include any one or a combination of: an ulnar artery; a radial artery; and a brachial artery. Accordingly, a highly effective location for the fabric portion is at the area of the arm at and near the crook of the elbow.

It is noted that veins, in comparison to arteries, are likely preferable “targets” for the cooling system, because the venous blood flows in the direction returning to the heart; regardless, exploiting superficial arteries may also be useful.

Some features of the present cooling garment may include that the (damp) fabric portion(s) is/are held snugly to the wearer’s body so as to be disposed above/on top of/adjacent at least one superficial blood vessel (prominent blood vessel) in a close fitting manner, to improve heat conduction from the at least one superficial blood vessel (prominent blood vessel) to the damp fabric portion prior or simultaneously to evaporation therefrom.

As understood from above, the terms “superficial blood vessel” and/or “prominent blood vessel”, or derivatives thereof, are used herein the specification and claims to denote any blood vessel that is adjacent the skin and, and by way of example, includes, but is not limited to, the blood vessels listed above. For clarification, visible spider veins, capillaries and varicose veins are not considered superficial blood vessels herein.

Potential advantages of the present cooling garment may include that only the area adjacent one or more superficial blood vessels of the clothing (which may be a fabric portion thereof) is wetted, thereby saving fluid/water; and/or the design can result in a reduced use (including rate of use) of fluid/water, which can potentially allow for an extended cooling period, i.e. extended period of comfortable exercise and lower loss of electrolytes; and/or result in a lower weight and more comfortable garment as the bladder (fluid reservoir) can be smaller. This specific and limited wetting is also potentially advantageous as there is less chance of the garment/clothing becoming loose, which could lessen the cooling effect as well as affecting the comfort of the wearer.

It is also noted that the fabric portion area of the clothing adjacent the at least one superficial blood vessel is preferably dampened and not saturated with cooling fluid, which also allows for reduced fluid use and lighter weight, as well as provides for improved evaporation and cooling. According to some designs of the garment, the flow of the cooling fluid can be regulated to achieve that goal.

Additionally, cooling is provided to the wearer’s blood stream, which can thus deliver a cooling effect throughout the wearer’s body and in particular to vital organs.

It is also noted that most superficial blood vessels are veins, in which blood flows back to the heart, which is thereby cooled, and can thus be significant in both the thermal comfort and health of the wearer; e.g. prevent heat stroke. The flow of the cooled blood through the body results in a natural process known as heat advection so that cooling the wear’s blood at a specific point(s) (i.e. superficial blood vessels) results in cooling other areas where the blood flows to.

As noted, veins are blood vessels that carry blood from various body parts back to the heart. Venous blood travels from the extremities back to the body’s core, where the blood absorbs heat, thus lowering core temperature, before flowing back to the extremities for heat loss. The lower the temperature of venous blood the more efficient the process of heat advection, the more heat the blood absorbs from the body’s core, and thus the more the blood lowers core temperature and reduces the chance of overheating.

Further, without limit to theory, it is believed that the lack of wetting of large areas of the clothing better allows for the natural evaporation of perspiration from the wearer’s body. Moreover, cooling of the body may result in less perspiration and therefore a drier shirt and increased comfort. Additionally, the design/configuration of the present garment may allow for a lower weight cooling garment as a result of reduced perspiration; and, compared to many prior art designs, limited wetting of the shirt by the system.

Depending on the particular design of the garment, the cooling fluid can help control the wearer’s temperature and via evaporative cooling and/or conductive cooling and/or advective cooling. The evaporative cooling may be enhanced if the wearer is moving, for example bicycle riding, or there is a breeze, whereby there is air flow over the dampened fabric portion.

### BRIEF DESCRIPTION OF THE DRAWINGS

The invention may be more clearly understood upon reading of the following detailed description of non-limiting exemplary embodiments thereof, with reference to the following drawings, in which:

FIG. 1 is a perspective front view of a cooling garment, including an article of clothing exemplified by a shirt, in accordance with embodiments of the present invention;

FIG. 2 is a perspective view of an exemplary article of clothing of the cooling garment, in the form of arm-sleeve sections; and

FIG. 3 is a perspective view of an exemplary fluid bladder of the cooling garment.

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

### DETAILED DESCRIPTION

Illustrative embodiments of the invention are described below. In the interest of clarity, not all features/components of an actual implementation are necessarily described.

The following detailed description of embodiments of the invention refers to the accompanying drawings referred to above. Dimensions of components and features shown in the figures are chosen for convenience or clarity of presentation and are not necessarily shown to scale. Wherever possible, the same reference numbers will be used throughout the drawings and the following description to refer to the same and like parts.

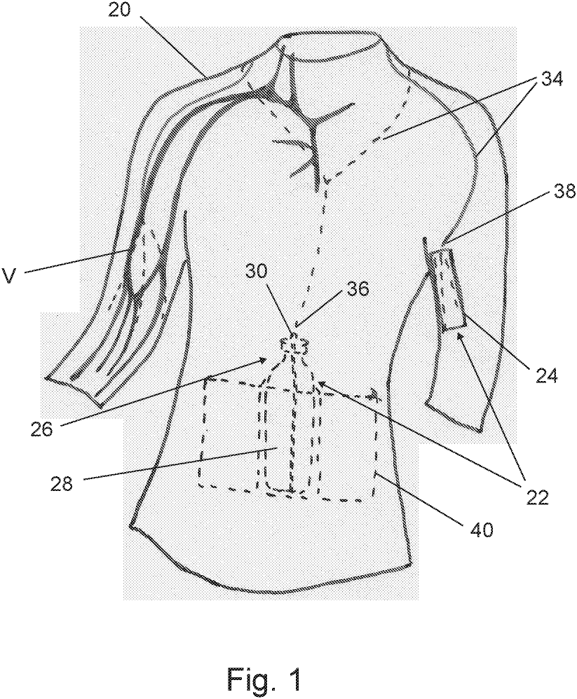

FIG. 1 shows a garment in accordance with embodiments of the present invention. The garment includes a clothing item or article of clothing 20 (exemplified by, but not limited to, a shirt, as illustrated); and a cooling system 22. Clothing 20 can be made of any suitable material including but not limited to cotton, polyester, or combinations/blends thereof. Clothing 20 may be configured so that the entire or essentially the entire clothing article fits snugly to the wear’s body, as is known per se, i.e. the clothing is made of a stretchy material so as to hold the material to the wearer’s skin. Clothing 20 may be configured so that only certain portions of clothing 20 fits snugly to the wear’s body, i.e. one or more certain portions of the clothing is made of a stretchy material so as to hold the material to the wearer’s skin, in particular to one or more portions of the clothing that are adjacent to one or more superficial blood vessels.

Clothing 20 includes a fabric portion 24 (illustrated on the left arm sleeve at the right side of FIG. 1) incorporated into or integrated within clothing 20, such as sewn into the clothing in place of otherwise existing/located material. Fabric portion 24 may alternatively be attached to the underside (inside / inwardly facing side) of clothing 20; or to the outer side (outside / outwardly facing side) of clothing 20. Fabric portion 24 is disposed so as to be adjacent to at least one superficial blood vessel V of the wearer, for example the axillary vein, and/or the basilic vein; and/or the median cubital vein; and/or the cephalic vein; and/or the subclavian vein. In other words, fabric portion 24 rests on top of at least a section of superficial blood vessel V, preferably touching the skin above the blood vessel.

Fabric portion 24 may be made of the same material as clothing 20 (illustrated on the right arm sleeve at the left side of FIG. 1) and as such may be indistinguishable from the remainder of the clothing. On the other hand, fabric portion 24 may be distinct and/or distinguishable relative to the remainder of clothing 20, for example being made of a different material; and/or having a different thickness; and/or having a different porosity; and or having a different tortuosity.

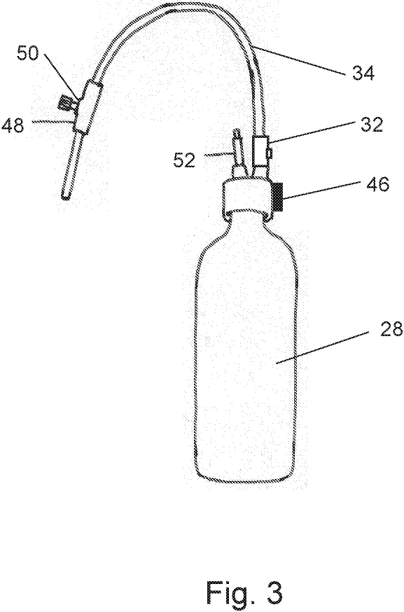

Cooling system 22 includes a fluid supply and dispensing apparatus 26, which includes at least one bladder 28 (or other such container(s), reservoir(s), etc., illustrated as a bottle) with a fluid outlet 30; a fluid pumping mechanism 32 (for example an electric pump schematically depicted in FIG. 3; a pressurized container; a syringe arrangement; a hand pump or a wicking material); and tubing 34. Although it is envisioned that the fluid used is water, it should be understood that any suitable fluid or combination could be used including other inorganic fluids, but also organic fluids, which may include one or more alcohols, any combination of the aforementioned; or even a humidified/moist gas that includes one or any combination of the aforementioned fluids.

Tubing 34 is connected at a proximal end 36 thereof to fluid outlet 30 and at one or more distal ends 38 the tubing dispenses fluid to fabric portion 24. As illustrated, tubing 34 may branch in order to deliver fluid to various locations where fabric portion 24 may be disposed. Tubing 34 can be standard polymeric tubing with a cylindrical profile or of a different configuration, as noted herein-below. It should be understood that there can be a plurality of bladders 28, with suitable tubing 34, mutatis mutandis.

Clothing 20 is typically configured to be snug to the wear’s body (and in particular at least in the area of fabric portion 24), whereby the fabric portion is held snugly to the body of the wearer. However, additionally or alternatively, clothing 20 may include a mechanism to ensure such snugness (e.g. an elastic strap, or a tightening scheme such as used in waist belts, or the like). Importantly, fabric portion 24 is disposed adjacent at least one of the major superficial blood vessels V of the wearer. Fabric portion 24 is designed to extend along a significant length or the entire length of at least one of superficial blood vessels V, in particular any portion of that/those blood vessel(s) that runs adjacent to the wearer’s skin. Fabric portion 24 may be formed in a strip-like shape, as exemplified, with margins on either side of superficial blood vessel V. For example, fabric portion 24 may be rectangular and be 10-15 cm long and 2-4 cm wide. If fabric portion 24 is integrated and made of the same material as clothing 20, distal end(s) 38 can be disposed so as to wet such an area; i.e. about 10-15 cm long and 2-4 cm wide, for example.

Distal end 38 (or tubing outlet 38) of tubing 34 wherefrom the fluid is delivered to fabric portion 24 is typically disposed at a generally upper portion of fabric portion 24, to take advantage of gravity so as to efficiently apply/dispense the fluid for efficient wetting throughout the fabric portion. It should be understood that adjacent a single superficial blood vessel V, there may be more than one tubing outlet 38 to help ensure wetting at the specifically desired length of superficial blood vessel V. Tubing outlet 38 may have a relatively flattened or streamlined profile, e.g. oval, semi-circle, crescent, fish-like, and so forth, in order to improve directed delivery of the fluid onto fabric portion 24 and prevent potential dripping or leakage outside of fabric portion.

It should be understood that as a result of the delivery of fluid to fabric portion 24, there will be a wicking of the delivered fluid to the fabric portion and evaporation therefrom whereby an evaporative cooling effect will be provided. Without limit to theory, it is believed that this cooling effect, due to the adjacency of fabric portion 24 to at least one superficial blood vessel, will cool superficial blood vessel V and the blood flowing therein. Thus, with the flow of that blood, at least some additional portion of the wearer’s blood stream will be cooled. Such an effect is significant as the wearer’s heart and other organs, muscles and so on may be cooled, thereby improving the wearer’s comfort and potentially preventing injury such as heat stroke.

It is also significant that the cooling garment delivers fluid directly to fabric portion 24 whereby the wearer’s blood stream is cooled, in contrast to additionally or merely cooling other/various portions of the wearer’s body, such as muscles or general body portions such as the chest, back, etc. As a result, without limitation to theory, it is believed that the present cooling garment provides efficient cooling to the wearer, including being able to use a relatively limited amount of fluid for cooling and/or (substantially directly) cooling the wearer’s blood stream, in comparison to known cooling garments.

Tubing distal end 38 may be relatively flat/streamlined (e.g. having a profile that is oval, semi-circle, crescent, or fish-like, and so forth), to additionally provide conductive cooling to fabric portion 24 in addition to the evaporative cooling and/or improve the delivery of fluid to fabric portion 24 and potentially improve the distribution of the fluid when dispensed. Further, all or at least a portion of tubing 34 may be streamlined. Without limitation to theory, such design can be advantageous as the fluid in streamlined tubing may provide pre-heating to the fluid (due to heat absorbed from the wearer’s body), which may promote evaporation thereof when the fluid is wicking in fabric portion 24. Additionally, a streamlined profile may improve wearer comfort, aesthetics, convenience of assembly, additional heat transfer (cooling). Without limitation to theory, in addition to the streamlined tubing design potentially improving the cooling effect to portions of the wearer’s body (in addition to cooling a superficial blood vessel), the streamlined tubing design can reduce heat transfer to ambient air and, as mentioned, advance the evaporation process by pre-heating the fluid eventually delivered to fabric portion 24.

It is reminded and emphasized that fabric portion 24 may be made of the same material as clothing 20 and can be completely indistinguishable therefrom; i.e. the clothing material is homogenous - the same throughout. It is important to note that fluid is still delivered to clothing 20 at a location appropriate to thereby provide cooling to superficial blood vessel(s) V. In other words, the specific feature of wetting an area of clothing 20 adjacent to at least one superficial blood vessel remains.

Clothing 20 may further include one or more bladder receiving pockets 40, or other such bladder holding/securing mechanism (e.g. hook and loop fasteners, corresponding hooks, fasteners, or the like). Additionally or alternatively, fluid supply and dispensing apparatus 26 may be configured to simply be held under clothing 20, for example due to the stretchiness of the clothing.

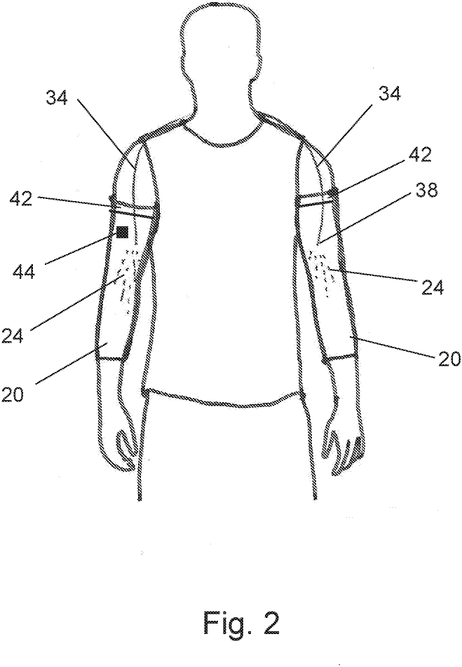

FIG. 2 illustrates clothing 20 configured as a pair of arm sleeve portions. Fluid supply and dispensing apparatus 26 may be configured to attach to the existing clothing (in a manner as noted above, for example), illustrated as a sleeveless shirt or “tank top” or sports top (e.g. a sports bra); or simply held under the sleeveless shirt or the like, as noted above.

Clothing 20 (whether constituted by a shirt, arm sleeve portions, or another article of clothing) may include an elastic strap 42 or the like (e.g. a strap-tightening mechanism such as used in swimming googles or waist belt, as noted above) to help maintain the clothing in place, and furthermore, to hold fabric portion 24 in its proper location; and moreover, snugly to the skin above superficial blood vessels V.

The garment may further include one or more sensors 44, for example located at clothing 20; however, additionally or alternatively at cooling system 22. Such sensors 44 may be configured to measure one or a number of parameters, for example, ambient temperature, and/or ambient humidity and/or other parameters that can be used to help determine if the wear should actuate fluid supply and dispensing apparatus 26 or change the rate of fluid delivery.

With reference to FIG. 3, additionally or alternatively, sensor(s) 44 may transmit data to a controller 46 to activate pump 32 (schematically illustrated at the outlet of bottle/bladder 28, for example a rotary positive displacement pump) or otherwise control the rate of fluid flow to fabric portion 24. Controller 46 may be configured to automatically operate fluid supply dispensing apparatus 26 (e.g. activate pump 32, in particular so as to provide a suitable fluid flow rate, for example using a variable speed drive; or in conjunction with a flow regulator 48). This automatic operation may be based on, for example, the ambient temperature - i.e. being above a pre-determined or a settable threshold temperature; and/or humidity or combination thereof, etc. Additionally or alternatively, fluid supply and dispensing apparatus 26 may be automatically controlled based on a different environmental or ambient parameter (which may be in combination with ambient temperature and/or humidity); or based on a biological parameter (i.e. of the wearer; for example heart rate and/or perspiration level). Controller 46 may operate a fluid flow valve 50 (or for example via flow regulator 48); or additionally or alternatively, the valve can be controlled manually by the wearer, preferably to cause fabric portion 24 to be damp and not fully saturated. If bladder 28 is configured as a rigid or semi-rigid vessel, a vent 52 may be provided. Alternatively or additionally, one or more bladder 28 may be a soft-walled container, whereby no vent would be necessary.

It should be understood that clothing 20, and in particular fabric portion 24, if a distinct material, may have a high surface area to promote wicking and/or evaporation of the fluid.

The present invention can additionally or alternatively be contemplated as a fluid supply and dispensing apparatus (e.g. fluid supply and dispensing apparatus 26) that is configured to be associated with an article of clothing, for example, as understood with reference to FIG. 3. Bladder 28 can be attached or otherwise held by existing clothing of the wearer, exemplified by a sleeveless shirt, and distal end(s) 38 of tubing 34 can be attached to an appropriate portion of clothing 20 (e.g. sleeve sections; wherein distal end(s) 38 can be disposed at a position or positions adjacent one or more superficial blood vessels). The fluid supply and dispensing apparatus may be used in combination with a clothing article configured to receive tubing 34 at a location adjacent one or more superficial blood vessels, as described above. The present invention can additionally or alternatively be contemplated as a clothing article configured to be associated with the fluid supply and dispensing apparatus, for example including a fixing mechanism for tubing 34 in order to provide fluid delivery to a location adjacent one or more superficial blood vessels, as described above. In other words, a wearer may own a number of clothing articles (e.g. shirts) and attach the fluid supply and dispensing apparatus.

Two tests were performed using a design including a full long-sleeve stretchy shirt and wherein water was pumped onto the bicep area of the arms adjacent superficial basilic, median cubital, cephalic veins, during a mountain bicycle ride. On days with similar weather, one week apart, the same bicycle rider rode the same 10.6 km off-road course. When not using the water supply and dispensing apparatus, the ride took 62.25 minutes at an average speed of 10.2 km/hour. When using the water supply and dispensing apparatus, the ride took 49.78 minutes at average speed of 12.8 km/hour.

It should be understood that the above description is merely exemplary and that there are various embodiments of the present invention that may be devised, mutatis mutandis, and that the features described in the above-described embodiments, and those not described herein, may be used separately or in any suitable combination; and the invention can be devised in accordance with embodiments not necessarily described above.

1. A cooling garment for cooling a wearer, the garment comprising:
an article of clothing; and
a cooling system associated with the article of clothing and comprising:
a fabric portion incorporated into or integrated within the article of clothing; and
a fluid supply and dispensing apparatus for supplying and dispensing fluid to the fabric portion and comprising (a) at least one fluid bladder for holding fluid; (b) a fluid pumping mechanism; and (c) tubing having a tubing outlet for conveying fluid from the bladder and dispensing the fluid from the tubing outlet onto the fabric portion,
wherein the clothing is configured to hold the fabric portion snugly to the body of the wearer and the fabric portion is disposed adjacent at least one superficial blood vessel so as to dispense the fluid specifically to the fabric portion to wet at least a portion of the fabric portion adjacent the at least one superficial blood vessel and not to other areas of the clothing whereby the fabric portion is cooled via evaporation.

2. The garment of claim 1, wherein the at least one superficial blood vessel includes any one or combination of: an axillary vein, a basilic vein; a brachial vein; a median cubital vein; a cephalic vein; and a subclavian vein.

3. The garment of claim 1, wherein the at least one superficial blood vessel includes any one or combination of: an ulnar artery; a radial artery; and a brachial artery.

4. (canceled)

5. (canceled)

6. (canceled)

7. (canceled)

8. The garment of claim 1, wherein the fabric portion is made of a material having a different tortuosity and/or different tortuosity than the remainder of clothing.

9. (canceled)

10. (canceled)

11. The garment of claim 1, wherein the tubing has at least one fluid outlet disposed at an upper end of the fabric portion.

12. (canceled)

13. The garment of claim 1, wherein the clothing is constituted by one or a pair of arm-sleeve portions of a shirt.

14. The garment of claim 1, wherein the fluid pumping mechanism comprises an electric pump.

15. The garment of claim 1, wherein the fluid pumping mechanism comprises a pressurized container.

16. The garment of claim 1, wherein the fluid pumping mechanism comprises a syringe arrangement.

17. The garment of claim 1, wherein the fluid pumping mechanism comprises a hand pump.

18. The garment of claim 1, wherein the fluid pumping mechanism comprises a wicking material.

19. The garment of claim 1, wherein the fluid pumping mechanism comprises a valve to control fluid flow.

20. (canceled)

21. The garment of claim 1, further comprising a sensor and a controller configured to automatically operate the fluid pumping mechanism based on an environmental parameter and/or a biological parameter.

22. The garment of claim 21, wherein the environmental parameter is ambient temperature and/or the biological parameter is skin temperature.

23. (canceled)

24. The garment of claim 1, wherein the clothing comprises a tightening mechanism to hold the fabric portion snugly to the wearer.

25. (canceled)

26. The garment of claim 24, wherein the tubing comprises a portion that is streamlined.

27. A method of cooling a wearer of a garment, the method comprising:
supplying and dispensing fluid to an article of clothing of the garment while being worn by a wearer;
wherein supplying and dispensing the fluid is specifically onto a portion of the clothing to wet at least a portion of the fabric portion that is held snuggly and adjacently to at least one superficial blood vessel whereby the fabric portion is cooled via evaporation.

28. The method of claim 27, comprising automatically controlling delivery of the fluid based on an environmental parameter and/or a biological parameter.

29. The method of claim 27, wherein automatically controlling delivery of the fluid is based on ambient temperature and/or skin temperature.

30. (canceled)

31. (canceled)
